(19) United States
(12) Patent Application Publication
(10) Pub. No.: US 2011/0288410 A1
(43) Pub. Date: Nov. 24, 2011

(54) METHODS AND SYSTEMS FOR DIAGNOSTIC ULTRASOUND BASED MONITORING OF HIGH INTENSITY FOCUSED ULTRASOUND THERAPY

(76) Inventors: Gavriel A. Speyer, Los Angeles, CA (US); Andrew A. Brayman, Edmonds, WA (US); Lawrence A. Crum, Bellevue, WA (US); Peter J. Kaczkowski, Seattle, WA (US)

(21) Appl. No.: 13/032,583

(22) Filed: Feb. 22, 2011

Related U.S. Application Data
(60) Provisional application No. 61/306,907, filed on Feb. 22, 2010.

(51) Int. Cl. A61B 8/00

(52) U.S. Cl. 600/438

(57) ABSTRACT

Methods and systems for monitoring the progress of high intensity focused ultrasound (HIFU) therapy use diagnostic ultrasound to identify temperature differentials using scatterer tracking between two backscattered radio frequency frames. The observed displacement of the scatterers may be combined with knowledge of the exposure protocol, material properties, heat transfer, and/or measurement noise to estimate heating, thermal dose, and temperature conditions resulting from the HIFU therapy.

### CROSS-REFERENCE TO RELATED APPLICATION

This application claims priority to U.S. Provisional Patent Application No. 61/306,907, filed Feb. 22, 2010, and incorporated herein by reference in its entirety.

### STATEMENT REGARDING FEDERALLY SPONSORED RESEARCH OR DEVELOPMENT

The following disclosure was made with government support under Grant Number 5R01CA109557 awarded by National Institutes of Health (NIH). The government has certain rights in the disclosure.

### TECHNICAL FIELD

The present technology is generally related to methods and systems for diagnostic ultrasound based monitoring of high intensity focused ultrasound therapy.

### BACKGROUND

Noninvasive therapies offer the ability to increase outpatient care and avoid complicated surgeries. High intensity focused ultrasound (HIFU) therapy is one such non-invasive modality. The ability to focus ultrasound and thereby confine treatment to a well defined focal volume has made HIFU therapy a promising technology. Unfortunately, left unmonitored, considerable prefocal and postfocal damage may be the inadvertent result of a particular therapy. At the other extreme, HIFU may not deliver the intended therapy to the target region. It is thus important to monitor the HIFU therapy.

Thermal dose has been recognized as a reliable clinical endpoint for cell necrosis, and in particular for HIFU therapy. To quantify the thermal dose administered at a given location, a time-temperature history is needed. Backscatter measured by diagnostic ultrasound provides time of flight changes in response to temperature changes in the material. These time of flight changes are expressed as either compressions or expansions of the diagnostic ultrasound image, to which the backscatter exhibits the greatest sensitivity. The technique of comparing two diagnostic ultrasound frames, one a reference frame taken before therapy and the other a treatment frame captured after therapy, and identifying the relative displacements between them is a proven method for forming estimates of temperature. Such cross-correlation techniques have been used to track the motion of small regions between the reference and treatment frames. The resulting displacement map, which catalogues these shifts nearly continuously, has been used to provide temperature estimates. However, such cross-correlation temperature measurement techniques have provided inaccurate values because, it is believed, the observed displacements are the product of a number of factors that confound conventional cross-correlation techniques.

### BRIEF DESCRIPTION OF THE DRAWINGS

FIG. 1 is a partially schematic illustration of a high intensity focused ultrasound (HIFU) system using diagnostic ultrasound to monitor the HIFU therapy configured in accordance with an embodiment of the disclosure.

FIGS. 2A and 2B are schematic illustrations of scatterers measured by a diagnostic ultrasound before and after the application of HIFU therapy in accordance with an embodiment of the disclosure.

### DETAILED DESCRIPTION

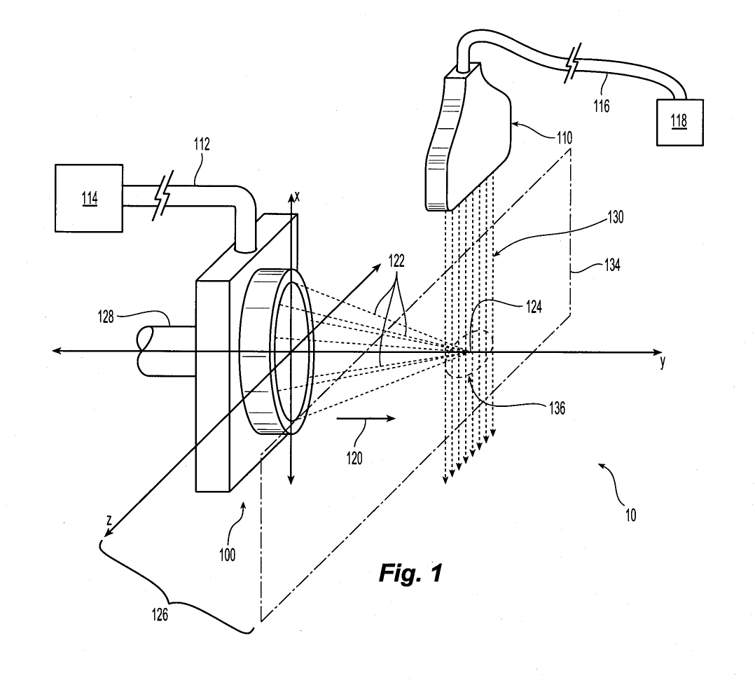

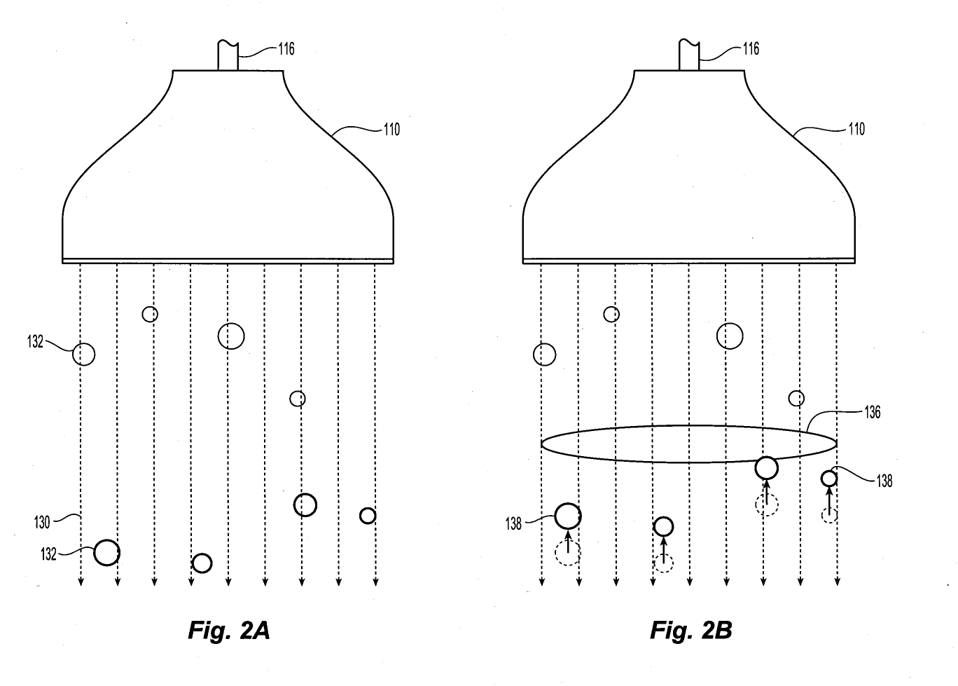

The present disclosure describes methods and systems for diagnostic ultrasound based monitoring of HIFU therapy. Specific details of several embodiments of the technology are described below with reference to FIGS. 1-2B. Other details describing well-known structures and systems often associated with HIFU therapy have not been set forth in the following disclosure to avoid unnecessarily obscuring the description of the various embodiments of the technology. Many of the details, dimensions, angles, and other features shown in the Figures are merely illustrative of particular embodiments of the technology. Accordingly, other embodiments can have other details, dimensions, angles, and features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 1-2B.

Certain aspects of diagnostic ultrasound and HIFU therapy can facilitate the evaluation and monitoring of the region treated in a HIFU therapy. The focusing of ultrasound confines the treated area, heat transfer constrains the rate at which this region changes, and measurement by diagnostic ultrasound interacts with the material in a deterministic way. This knowledge about treatment and monitoring allows diagnostic ultrasound measurements to be combined efficiently to produce a more concise description of therapy. By reducing the degrees of freedom in diagnostic ultrasound measurements through physical modeling, fundamental limits can be obtained for diagnostic ultrasound based monitoring as well as estimators for the delivered therapy. Estimates of the heating rate during the exposure interval can be provided as a linear combination of modes of an observation kernel, determined by the manner in which the diagnostic ultrasound observes therapy when only the heating rate is unknown.

Each mode of therapy observation, termed a heating mode, when individually applied as a source of therapy, induces a particular pattern of displacements, termed the displacement mode, between the reference and treatment frames. The displacement modes are linearly independent, and each is uniquely related to the respective heating mode. Since therapy estimation is performed in the space of displacement—by aligning at least two frames—the process is most aptly described as displacement mode analysis (DMA). DMA assumes that the relative displacements between frames are linearly related to the prevailing temperature in the material. The components central to this method are the time of flight model for diagnostic ultrasound measurement, the treatment protocol, the relevant mechanics of heat transfer, and the statistical measurement model affecting diagnostic ultrasound observations. These combine to produce DMA as a natural framework for therapy monitoring, when using maximum likelihood as the performance index.

Some embodiments described herein involve the use of a modal analysis of a particular Kernel (see U.S. Provisional Patent Application No. 61/306,907, filed Feb. 22, 2010, at page 34, Eq. (12)), whose modes correspond to distinct HIFU modulations. As noted previously, App. No. 61/306,907 is incorporated herein by reference in its entirety. The observability of these modes, as provided by the use of time-of-flight changes, is given by the corresponding eigenvalue. The reciprocal of this quantity is proportional to the variance of the coefficient estimate for this mode. Among the identified modes is a therapy which can be resolved with the highest resolution, having the largest eigenvalue, and provides the ideal modulation for diagnostic ultrasound monitoring.

Several embodiments of the methods and systems described herein are directed to the use of imaging with diagnostic ultrasound to characterize treatment conditions during HIFU therapy. More specifically, various aspects of the present disclosure use diagnostic ultrasound imaging to monitor and estimate the spatial distribution of temperature in tissue in response to HIFU therapy. In one embodiment, for example, the temperature within tissue targeted with a HIFU treatment protocol can be estimated by evaluating the position of backscattering observed in ultrasound images of the tissue. The backscattering observed in ultrasound images provides a distribution of observable points (backscatterers) positioned within an otherwise ultrasound-transparent or nearly-transparent medium such as certain types of tissue. The displacement of the backscatterers over a period of time provides information about a change in the temperature of the medium.

By comparing ultrasound images between a first frame preceding a round of HIFU treatment and a second, different frame following the treatment, the backscattering observed in the first and second frames can be evaluated to identify distortions in the images. The distortion observed in the backscattered ultrasound images is related to the bio-heat transfer equation (BHTE), the HIFU exposure protocol, the HIFU beam pattern, and the specific material properties of the target tissue. The observed backscattering and distortion, information regarding the HIFU treatment protocol, and information regarding the properties of the targeted tissue can be evaluated to provide information regarding the temperature of the targeted tissue and the area surrounding the targeted tissue. In other embodiments, the same observations and information can be further evaluated to providing information regarding changes in the temperature of the targeted tissue, the heat rate and energy levels associated with the HIFU therapy, and the safety and effectiveness of the HIFU therapy.

FIG. 1 is a partially schematic illustration of a HIFU system 10 using diagnostic ultrasound to monitor the HIFU therapy configured in accordance with an embodiment of the disclosure. The HIFU therapy system 10 of FIG. 1 includes a HIFU transducer 100 and a diagnostic ultrasound imaging probe 110. The HIFU transducer 100 is operationally connected by a cable 112 to a HIFU control system 114 (shown schematically) that permits an operator of the system (not shown) to provide power to and control operation of the HIFU transducer 100. The imaging probe 110 is operationally connected by a cable 116 to a diagnostic ultrasound control system 118 (shown schematically) to provide power to and control of the imaging probe 110. The HIFU control system 114 and the diagnostic ultrasound control system 118 can be operationally connected to each other to provide communication between the two systems, be operationally joined to provide a single or commingled system, or be each operationally connected to another master control system (not shown) controlling or monitoring the operation of both systems.

As shown in FIG. 1, the HIFU transducer 100, when operational, provides acoustical energy in a direction 120 along a conical path 122 that forms a focus 124 at a focal distance 126 from the HIFU transducer 100. The HIFU transducer 100 can be, for example, a Model SU-107 transducer marketed by Sonic Concepts of Woodinville, Wash., USA. In other embodiments, however, a variety of other suitable HIFU transducers may be used. The HIFU transducer 100 is configured to provide a controlled level of acoustic energy to the focus 124. A framing system 128 (shown in part) can be used to control the position and movement of the HIFU transducer 100 and focus 124 relative to a desired position selected for HIFU therapy. The framing system 128 also provides three-axis positioning and movement of the HIFU transducer 100 with the use of a position controller component of the HIFU control system 114. As shown in FIG. 1, the direction 120 of the acoustical energy is along a y-axis defined by the HIFU transducer 100 and the focus 124 which is intersected by the y axis. The framing system 128, when supporting the HIFU transducer 100, defines additional axes, such as an x-axis defining vertical position and motion of the transducer, and a z-axis defining horizontal position and motion of the transducer. The x-axis, y-axis, and z-axis together provide a coordinate system by which the location of the focus 124 can be determined.

The diagnostic ultrasound imaging probe 110 is disposed to direct an ultrasound signal 130 from the probe 110 along an imaging plane 134 at least approximately parallel with a plane defined by the x-axis and z-axis of the HIFU transducer 100. In this way, the ultrasound signal 130 intersects with the focus 124 and intersects the y-axis at a right angle. As shown and described in greater detail below with reference to FIG. 2A, the ultrasound signal 130, in part, passes through or is at least partially absorbed by the medium through which it passes and, in part, reflects from backscatter sources 132 back towards the imaging probe 110 that contains a sensor that receives the reflected ultrasound signal 130. The received signal is processed by the ultrasound control system 118 to provide an image of the area traversed by the reflected ultrasound signal 130. The framing system 128 can support the imaging probe 110 so as to provide alignment between the ultrasound signal 130 and the focus 124. Alternatively, the imaging plane 134 can be located in the y-z plane aligned with the HIFU transducer 100.

In operation, the HIFU transducer 100 provides acoustic energy to the focus 124 at an intensity sufficient to produce a heating effect consistent with HIFU therapy and, in accordance with a HIFU therapy protocol, the imaging probe 110 provides an image of the area surrounding the focus 124 along the imaging plane 134. While administering the HIFU therapy, the HIFU transducer 100 can be moved in a direction that disposes the focus 124 along a path that traverses an area to be treated by HIFU therapy. For example, the focus 124 can be moved along a length of a lesion (not shown) so as to provide HIFU therapy to the lesion with an intensity that provides a therapeutic effect.

In an embodiment, the imaging probe 110 is configured to capture two images or frames in the focal plane of the HIFU transducer 100. The first frame precedes HIFU treatment and the second frame follows HIFU treatment. Observed differences, primarily due to the apparent displacement of backscatter sources 132, observed when comparing the two frames, are the result of time of flight changes experienced in regions of elevated temperature located at the focus 124. It is believed that an elevated temperature, relative to the surrounding environment, at or near the focus 124 changes the speed of sound of the medium or tissue through which the reflected ultrasound signal passes, thereby causing the time of flight for the observed area to change. The temperature change is believed to be due to the heating introduced during HIFU therapy.

FIGS. 2A and 2B are schematic illustrations of scatterers measured by a diagnostic ultrasound system (e.g., the diagnostic ultrasound 110 of FIG. 1) before and after the application of HIFU therapy in accordance with an embodiment of the disclosure. More specifically, referring first to FIG. 2A, in operation the ultrasound signal 130 is emitted from the imaging probe 110 towards the focus 124, and a portion of the signal 130 reflects back from backscatter sources 132 towards the imaging probe 110. The diagnostic ultrasound control system 118 (FIG. 1) subsequently provides an ultrasound image of the area surrounding the focus that displays the observed backscatter sources 132 disposed at specific locations within the observed tissue. In FIG. 2A, which schematically represents the tissue prior to HIFU treatment in the first frame, a portion of the ultrasound signal 130 continues past the focus 124 to reflect from backscatter sources 132 located past the focus 124.

Referring next to FIG. 2B, which schematically represents the tissue after HIFU treatment in the second frame, the ultrasound signal 130 reaches the focus 124 and encounters a treated region 136 that presents a different speed of sound as compared to the surrounding tissue or as compared to the tissue prior to treatment. A portion of the ultrasound signal 130 passes through the treated region 136 and reflects back from backscatter sources 132 located past the treated region 136. The reflected ultrasound signal 130 travels towards the imaging probe 110 and, again, passes through the treated region 136 a second time. It is believed that the change in the speed of sound of the treated region 136 alters the ultrasound signal 130 as it passes through the treated region 136 so as to cause the diagnostic ultrasound control system 118 (FIG. 1) to provide an ultrasound image that shows the backscatter sources 132 located past the treated region as being displaced from an original position observed prior to the HIFU treatment. It is also believed that, to some degree, the HIFU treatment causes heating in the treated region 136 which, in turn, can actually displace the backscatter sources 132 subject to the heating. The displacement is displayed in the second frame as a compression towards the imaging probe 110 and is believed to be a result of an increased sound speed in the treated area.

The observed displacement of the displaced backscatter sources is believed to be related to the temperature of the treated region. However, it is further believed that the imaging data obtained from the displaced backscatter sources can be improved by accounting for several factors, including the relative positioning of the diagnostic ultrasound imaging probe and HIFU transducer, the treatment duration, the HIFU transducer radiation pattern, the motion of the HIFU transducer, and the acquisition characteristics of the diagnostic ultrasound equipment.

Several embodiments of the present disclosure are directed to statistical formulation of diagnostic ultrasound based HIFU monitoring with which the impact of these factors on the uncertainty in estimating the heating delivered to the focus can be quantitatively assessed. Such a characterization is believed to be useful for temperature estimation because the temperature distribution within the observed tissue is derived from the applied heating through the bio-heat transfer equation (BHTE), which allows temperature estimates to be characterized as well. The robustness of the heating estimation can be evaluated using multiple factors, such as the maximum possible power that can be delivered to the focus, the maximum possible temperature prevailing within the tissue when the second frame is captured, and the maximum possible relative displacement of backscatter sources between the two frames. In an embodiment, an estimation algorithm can provide an estimation of the heating in at least approximately real time. In further embodiments, the performance of the HIFU therapy system using the estimation algorithm can be evaluated analytically so as to provide verification of the aforementioned factors.

As described above, the imaging data obtained from the diagnostic ultrasound and displaced backscatter sources can be improved by evaluating additional factors when assessing the effect the HIFU therapy has upon the treated region. One such factor are the relative positions of the energy producing components to the energy receiving components of the HIFU therapy system, such as the diagnostic ultrasound imaging probe or the HIFU transducer. More specifically, by accounting for the geometry and orientation between the HIFU transducer and the imaging probe relative to each other and the treated region, the calculation of the heating effect at the treated region provide by the frame comparison can be improved. Another factor that can be incorporated into the analysis of the frame comparison is the duration of the treatment, which includes a quantification of the applied energy and any variation or interruption in the application of energy to the treated region. Yet another factor that can be incorporated into the frame comparison analysis is the emission pattern of the HIFU transducer, which includes an assessment of the shape of the energy emitted by the HIFU transducer. Still another factor that can be incorporated into the frame comparison analysis is the movement of the HIFU transducer and imaging probe during the treatment protocol, which includes an assessment of any velocity and rotation that may influence the energy emitted by the HIFU transducer or detected by the imaging probe. Still yet another factor that can be incorporated into the frame comparison analysis are the acquisition characteristics of the diagnostic ultrasound equipment used during the treatment protocol, which includes an assessment of any inefficiencies or losses with the acquisition characteristics.

The assessment of these additional factors, among others appropriate for the particular HIFU therapy system, can be incorporated into the frame comparison analysis to provide an improved value for the temperature of the treated region and the temperature changes in and around the treated region. It was determined that consideration of these additional factors provided temperature values that were within five times more sensitive than methods limited to frame comparison.

Another feature of several embodiments of the techniques described herein is that the disclosed techniques can be used for reconstructing the delivered therapy (i.e., heating of the course of treatment). For example, the disclosed technology can use the temperature field (e.g., prevailing when the second RF frame is captured) to form an estimate of the administered heating, and thereby provide an accurate estimate of the thermal dose.

Additional embodiments improves the calculation of the heating effect by identifying the best basis with which to estimate the heating, such that coefficient estimates considered in calculating the heating rate have minimum variance. For example, the heating rate can be represented over the interval (t_i,t_f) using an expansion of the form

$$q ( t ) = {\sum }_{n} {q}_{n} {φ}_{n} ( t )$$

Both the functions φₙ(t) and the variance of coefficient estimates var (qₙ) are determined a priori from the measurement noise, scattering power of the medium, relative positions of diagnostic and therapeutic transducers, duration of the heating interval, and the solution to the heat equation. This collection of known information is subsumed into the heating kernel K, as discussed in detail in U.S. Provisional Patent Application No. 61/306,907, filed Feb. 22, 2010, and incorporated herein by reference in its entirety. By identifying these waveforms, the first practical result is that the wave function φₙ(t) associated with the minimum variance var (qₙ) is the one which should be used for modulation of the HIFU, since it can be monitored with the greatest accuracy. More generally, the heating modes can give rise to orthogonal displacement modes, which determine the movement of scatterers in the frame in response to a given heating mode, that are independent, like the heating modes, and hence their contributions uniquely reflect those of the separate heating modes. In this way, heating is identified from the pattern of scatterer displacement.

It will be appreciated that the embodiments described herein can be modified to use alternative technologies. For example, instead of using diagnostic ultrasound to monitor the treated region, other monitoring or measuring technologies can be used (e.g., magnetic resonance imaging, etc.). In another example, instead of using a HIFU transducer to impart energy to a focus, other energy systems can be used to provide energy to a targeted site, such as a radiation-emitting material or devices. In still another example, the embodiments described herein can be used to monitor and measure temperature in materials other than tissue. For example, the monitoring and measuring systems described herein can be used to evaluate cavities within a human or animal body, structures holding a fluid such as a stomach, or devices, drugs, or chemical contained with a human or animal body. In yet another example, the same monitoring and measuring systems can be modified to evaluate the internal temperature of a non-biological material. In another example, more than two frames can be used for the frame comparison, providing a plurality of sequential image frames that are collected and used in the analysis, which is believed to further improve the accuracy of the estimates of temperature rise.

Various embodiments of the technology described herein can be implemented in suitable computing environments. Although not required, aspects and embodiments of the technology will be described in the general context of computer-executable instructions, such as routines executed by a general-purpose computer, e.g., a server or personal computer. Those skilled in the relevant art will appreciate that the described technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers, and the like. The described technology can be embodied in a special purpose computer or data processor that is specifically programmed, configured, or constructed to perform one or more of the computer-executable instructions explained herein. Indeed, the term “computer,” as used generally herein, refers to any of the above devices, as well as any data processor or any device capable of communicating with a network, including consumer electronic goods such as handheld devices or other electronic devices having a processor and other components, e.g., network communication circuitry.

The described technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines may be located in both local and remote memory storage devices. Aspects of the technology described herein may be stored or distributed on computer-readable media, including magnetic and optically readable and removable computer discs, magnetic cassettes, tape drives, flash memory cards, digital video disks (DVDs), Bernoulli cartridges, RAMs, ROMs, smart cards, stored as firmware in chips (e.g., EEPROM chips), etc. Indeed, any medium for storing or transmitting computer-readable instructions and data may be employed, including a connection port to or node on a network such as a local area network (LAN), wide area network (WAN), or the Internet. Alternatively, aspects of the disclosure may be distributed electronically over the Internet or over other networks (including wireless networks). Those skilled in the relevant art will recognize that portions of the described technology may reside on a server computer, while corresponding portions reside on a client computer. Data structures and transmission of data particular to aspects of the described technology are also encompassed within the scope of the described technology.

From the foregoing it will be appreciated that, although specific embodiments of the technology have been described herein for purposes of illustration, various modifications may be made without deviating from the spirit and scope of the technology. Further, certain aspects of the new technology described in the context of particular embodiments may be combined or eliminated in other embodiments. For example, in the embodiments illustrated above, various combinations of monitoring and measuring systems may be combined into different systems. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A method, comprising:
treating target tissue of a subject with high intensity focused ultrasound (HIFU) therapy, wherein the HIFU therapy is delivered from a transducer;
using a diagnostic ultrasound device to capture a first ultrasound frame in a focal plane of the transducer before applying the HIFU therapy to a target tissue, and a second ultrasound frame in the focal plane of the transducer after applying the HIFU therapy to the target tissue; and
determining the temperature of the target tissue based on a comparison of data from the first ultrasound frame with data from the second ultrasound frame, and a factor derived from one or more of a distribution pattern of energy emitted from the transducer, a heat transfer constraint of the target tissue, a scattering pattern observed in the target tissue, and an interaction between the transducer and the diagnostic ultrasound device.

2. The method of claim 1, wherein determining the temperature of the target tissue occurs in at least approximately real time with the HIFU therapy of the target tissue.

3. The method of claim 1, wherein the comparison of data from the first frame with data from the second frame comprises comparing the relative displacement of scatterers between the first and second frames.

4. The method of claim 1, wherein the capture of the first frame and the second frame comprises capturing the first and second frames with a scanhead of the diagnostic ultrasound positioned to measure backscattering in the focal plane of the transducer.

5. The method of claim 1, wherein the factor further comprises an orientation between the transducer and the diagnostic ultrasound device.

6. A method, comprising:
receiving a first value representing a displacement of a backscatter source;
receiving a second value representing a factor derived from one or more of a distribution pattern of a first energy emitting source, a heat transfer constraint of a material, a scattering pattern observed in the material, and an interaction between the first energy emitting source and a second energy emitting source; and
determining a temperature based on a relationship between the first and second value.

7. The method of claim 6, wherein a first energy emitted from the first energy emitting source traverses a plane defined by the second energy emitted from the second energy emitting source.

8. The method of claim 7, wherein the first energy traverses the plane at or near a right angle.

9. The method of claim 6, wherein a first energy emitted from the first energy emitting source is configured to define a focus disposed in a plane defined by a second energy emitted from the second energy emitting source.

10. The method of claim 6, further comprising:
determining a temperature change based on another relationship between the first and second value.

11. A HIFU therapy system, comprising:
a transducer disposed to emit a first energy to a focus of the transducer;
an ultrasound emitting device disposed to emit a second energy to the focus of the transducer;
an ultrasound detecting device disposed to receive the second energy reflecting from a backscatter source; and
a temperature determining device configured to determine a temperature at the focus based on a relationship between a position of the backscatter source and a factor derived from one or more of a distribution pattern of the first energy, a heat transfer constraint disposed at the focus, a scattering pattern of the backscatter source, and an interaction between the transducer and the ultrasound emitting device.

12. The system of claim 11, wherein the transducer engages a frame disposed at a fixed distance from the ultrasound emitting device or the ultrasound detecting device.

13. The system of claim 11, wherein the transducer is movably disposed while emitting the first energy.

14. The system of claim 13, wherein the ultrasound emitting device or the ultrasound detecting device is disposed to move in a direction that is at least in part based on a movement of the transducer.

15. The system of claim 11, wherein the temperature determining device is further configured to determine a temperature change at the focus.

16. A physical computer-readable storage medium having stored thereon, computer-executable instructions that, if executed by a computing system, cause the computing system to perform operations, comprising:
treating a target tissue of a subject with high intensity focused ultrasound (HIFU) therapy, wherein the HIFU therapy is delivered from a transducer;
using a diagnostic ultrasound to capture a first ultrasound frame in a focal plane of the transducer before the HIFU therapy of the target tissue, and a second ultrasound frame in the focal plane of the transducer after the HIFU therapy of the target tissue; and
determining the temperature of the target tissue based on a comparison of data from the first ultrasound frame with data from the second ultrasound frame, and a factor derived from one or more of a distribution pattern of energy emitted from the transducer, a heat transfer constraint of the target tissue, a scattering pattern observed in the target tissue, and an interaction between the transducer and the diagnostic ultrasound device.

17. The computer-readable storage medium of claim 16, wherein determining the temperature of the target tissue occurs in at least approximately real time with the HIFU therapy of the target tissue.

18. The computer-readable storage medium of claim 16, wherein the comparison of data from the first frame with data from the second frame comprises comparing the relative displacement of scatterers between the first and second frames.

19. The computer-readable storage medium of claim 16, wherein the capture of the first frame and the second frame comprises capturing the first and second frames with a scanhead of the diagnostic ultrasound positioned to measure backscattering in the focal plane of the transducer.

20. The computer-readable storage medium of claim 16, wherein the factor further comprises an orientation between the transducer and the diagnostic ultrasound device.
